(19) United States
(12) Patent Application Publication
(10) Pub. No.: US 2012/0253387 A1
(43) Pub. Date: Oct. 4, 2012

(54) DEVICE SYSTEM AND METHOD FOR TISSUE ACCESS SITE CLOSURE

(75) Inventors: Eyal Teichman, Hod-HaSharon (IL); Boaz Harari, Haifa (IL)

(73) Assignee: APICA CARDIOVASCULAR IRELAND LIMITED, Galway, County Galway (IE)

(21) Appl. No.: 13/513,587

(22) PCT Filed: Dec. 1, 2010
(86) PCT No.: PCT/IL2010/001007

§ 371 Date: Jun. 4, 2012

Related U.S. Application Data
(60) Provisional application No. 61/265,798, filed on Dec. 2, 2009.

(51) Int. Cl. A61B 17/03

(52) U.S. Cl. 606/213

(57) ABSTRACT

A system for closure of a vascular access site and a device for closure are provided. The system includes a radially expandable device sized and configured for positioning within a blood vessel and a delivery catheter. The catheter is designed for delivering the radially expandable device through the vascular access site and expanding the radially expandable device in a position that spans the vascular access site, thereby at least partially closing the vascular access site.

### FIELD AND BACKGROUND OF THE INVENTION

The present invention relates to a device, system and method which can be used to partially or fully close tissue access sites and in particular, access sites in tubular vessels such as blood vessels (vascular access sites).

More than five million percutaneous interventions are performed annually in the United States, involving femoral artery catheterization for diagnostic or therapeutic purposes.

Most procedures are performed through small sheath access sites (5-8 F) and thus closure of such access sites can be effected using manual or mechanical compression for 15-30 minutes, typically combined with an extended bed-rest of three to six hours.

However, manual compression can cause patient discomfort, and is time- and resource-intensive, and as such, a need for quicker, more patient compatible closure has led to the introduction of closure devices in the early 1990s. Since then, vascular closure systems have been simplified to provide wider patient access to a range of vascular procedures. Now available from many sources, these devices shorten procedure times, allow patients to ambulate earlier, minimize bleeding and possibly reduce costs associated with hospital care.

At present there are dozens of devices on the market or at various stages of development, such devices employ sutures, patches, glue, coagulants and/or staples or a source of energy to effectively seal access sites post procedure.

Although these devices were specifically designed for closure of small access sites (<10 F), there have been attempts since the late 90s to utilize suture closure devices (specifically the Sutura™ and Perclose™ devices) in large bore access sites >18 F, illustrating at least a limited need for ‘automated’ closure of large access sites. Large bore access site closure is typically effected via manual suturing of an exposed artery and thus requires presence of a specialist while being time consuming as well as more invasive.

The studies performed to date illustrate that closure of access sites less than 18 F in size via such devices is effective and highly successful, whereas closure of larger bore access sites (e.g. 22 F) is less effective.

Although at present the number of procedures effected through large bore access sites is small, current trends anticipate that the number of such procedures will rise in the future and although a concomitant reduction in sheath sizes might also take place, such reduction will still place average sheath size at over 18 F.

While reducing the present invention to practice, the present inventors have devised an access site closure system which enables reliable closure of large bore access sites while providing re-access if necessary.

### SUMMARY OF THE INVENTION

According to one aspect of the present invention there is provided a system for closure of a vascular access site comprising: (a) a radially expandable device sized and configured for positioning within a blood vessel; and (b) a delivery catheter for (i) delivering the radially expandable device through the vascular access site; and (ii) expanding the radially expandable device in a region within the blood vessel spanning the vascular access site, thereby at least partially closing the vascular access site.

According to further features in preferred embodiments of the invention described below, the radially expandable device is a stent graft.

According to still further features in the described preferred embodiments the radially expandable device is self expanding.

According to still further features in the described preferred embodiments the radially expandable device includes two opposing expandable rings interconnected via a sleeve.

According to still further features in the described preferred embodiments each of the two opposing expandable rings is less than 10 mm in width.

According to still further features in the described preferred embodiments the sleeve is fabricated from a tubular sheet of ePTFE.

According to still further features in the described preferred embodiments the delivery catheter includes a mechanism for expanding the radially expandable device.

According to still further features in the described preferred embodiments the radially expandable device includes a balloon and the mechanism is an inflation mechanism.

According to still further features in the described preferred embodiments the radially expandable device is compressed within a sheath and the mechanism is a sheath removal mechanism.

According to still further features in the described preferred embodiments the radially expandable device includes an aperture in a side wall along a length thereof, the aperture being for providing the delivery catheter access to a lumen of the expandable tubular body.

According to still further features in the described preferred embodiments the delivery catheter and the radially expandable device form a t-shape when the radially to expandable device is positioned in the blood vessel.

According to still further features in the described preferred embodiments the delivery catheter engages the radially expandable device through the aperture, such that a guide wire can be threaded through the delivery catheter though a lumen of the radially expandable device and into a lumen of the blood vessel.

According to still further features in the described preferred embodiments the aperture is capable of at least partially closing when the delivery catheter is disengaged therefrom.

According to another aspect of the present invention there is provided a device for closure of a vascular access site comprising a radially expandable tubular body having an aperture in a side wall along a length thereof, the aperture being for providing a delivery catheter access to a lumen of the expandable tubular body.

According to still further features in the described preferred embodiments the radially expandable tubular body includes two opposing expandable rings interconnected via a sleeve.

According to still further features in the described preferred embodiments each of the two opposing expandable rings is less than 10 mm in width.

According to still further features in the described preferred embodiments the sleeve is fabricated from a tubular sheet of ePTFE.

According to another aspect of the present invention there is provided a method of at least partially closing a vascular access site comprising: (a) positioning a radially expandable device having an aperture in a side wall along a length thereof within a blood vessel through the vascular access site; and (b) aligning the aperture with the access site and expanding the radially expandable device within the blood vessel at a region spanning the vascular access site; and (c) at least partially closing the aperture thereby at least partially closing the vascular access site.

According to still further features in the described preferred embodiments steps (a) and (b) are effected using a delivery catheter.

According to still further features in the described preferred embodiments the delivery catheter engages the radially expandable device through the aperture, such that a guide wire can be threaded through the delivery catheter though a lumen of the radially expandable device and into a lumen of the blood vessel.

According to still further features in the described preferred embodiments the delivery catheter includes a mechanism for expanding the radially expandable device.

According to still further features in the described preferred embodiments the radially expandable device is disposed over a balloon and the mechanism is an inflation mechanism.

According to still further features in the described preferred embodiments the radially expandable device is compressed within a sheath and the mechanism is a sheath removal mechanism.

According to still further features in the described preferred embodiments the delivery catheter and the radially expandable device form a t-shape when the radially expandable device is positioned in the blood vessel.

According to still further features in the described preferred embodiments the aperture is capable of at least partially closing when the delivery catheter is disengaged therefrom.

According to yet another aspect of the present invention there is provided a system for delivering a stent-graft to a body lumen comprising: (a) a radially expandable device sized and configured for positioning within the body lumen, the radially expandable device including two opposing expandable rings interconnected via a sleeve having an aperture in a side wall along a length thereof; and (b) a delivery catheter including an internal sheath and an external sheath, wherein the radially expandable device is packable within the external sheath with a first ring of the two opposing expandable rings being disposed around the internal sheath and the second ring of the two opposing expandable rings being disposed adjacent to the internal sheath.

According to still further features in the described preferred embodiments the radially expandable device is packable within the external sheath with the internal sheath inserted through the aperture.

The present invention successfully addresses the shortcomings of the presently known configurations by providing a closure device and system that enables rapid and easy closure of, for example, a vascular access site while also enabling subsequent vascular reentry.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1-8 illustrate one embodiment of the present system through stages of deployment and stent-graft positioning.

FIG. 9 illustrates one embodiment of a stent-graft constructed in accordance with the teachings of the present invention.

FIG. 10 depicts the setup used for testing feasibility of the present approach.

FIGS. 11A-E illustrate the steps in deploying the tubular element (representing the stent-graft) out of the delivery catheter assembly and into a silicone tube representing an artery.

FIGS. 12A-D illustrate a platform used to test blood flow through an artery having an access site closed using the device of the present invention. FIG. 12B illustrates the silicone tube ‘artery’ portion of the platform shown in FIG. 12A showing to the access site formed therein via a cross-shaped incision. FIG. 12C illustrates the device of the present invention (stent-graft, schematically illustrated in FIG. 13D) positioned within the silicon tube (FIG. 12B) of the testing platform. FIG. 12D illustrates inward collapse of one of the stent-like rings of the present device due to application of external pressure.

FIGS. 13A-C illustrate another embodiment of the present system through stages of deployment and stent-graft positioning.

FIG. 13D illustrates the embodiment of the present device formed from two-opposing rings connected via a tubular sheet.

### DESCRIPTION OF THE PREFERRED EMBODIMENTS

The present invention is of a system which can be used to partially or fully close a vascular access site. In one embodiment, the present invention employs a stent-graft which can be positioned in a lumen of a blood vessel across the access site thereby sealing/reducing the access site hole and preventing blood leakage therefrom.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Percutaneous access to coronary and other major blood vessels is slowly replacing open surgical access and is driving a need for accessory technologies such as access site closure systems.

Although small access sites (<8-10 F) can be effectively closed using existing technologies, solutions for effective closure of large access sites (>10-12 F) are still lacking.

Since existing approaches co-apt tissue edges surrounding the access site hole, use of such approaches in closure of large access sites can lead to a substantial reduction in blood vessel diameter as well as vessel kinking. As a result, the present inventors have postulated that effective closure of large access sites requires a new approach rather than modification of existing approaches.

Thus, according to one aspect of the present invention there is provided a system for closure of a vascular access site.

As used herein, the phrase “vascular access site” refers to the tissue site through which vasculature of a subject is accessed. The access site can be formed in any blood vessel suitable for access. Examples include the femoral artery, the radial artery and the subclavian artery.

The system of the present invention includes a radially expandable device (also referred to herein as the “device”) which is sized and configured for positioning within a blood vessel and a delivery catheter for delivering the radially expandable device through the vascular access site and positioning it such that it spans the vascular access site thereby partially or fully blocking the access site hole.

The radially expandable device can be any device that can be expanded within the blood vessel lumen to apply a force onto the inner wall of the blood vessel surrounding the access site hole.

In that respect, when expanded within the lumen, the radially expandable device can expand to assume a substantially tubular configuration with, for example, closed (O-shaped cross section) or open (C-shaped cross section) profiles.

Several configurations of the radially expandable device can be used with the present invention, including, but not limited to, closed or open tubes fabricated from rolled sheets, coated/covered wire frames (e.g. stent-grafts) or tubular sheets interconnected via two opposing stent-like rings.

The radially expandable device of the present invention can be fabricated by laser cutting a polymeric or alloy (e.g. Nitinol, Cobalt Chromium or stainless steel) tube, or by braiding or knitting polymeric or alloy wires over a mandrel to form a tubular structure or portions thereof.

The tubular structure can be fabricated as two opposing expandable rings connected via rods or struts or interconnected via the tubular graft material described below. Each of the rings can have a width of 5-20 mm, preferably, 5-10 mm, most preferably 6-8 mm (along the axis defining the length of the device).

The radially expandable device can be pre-shaped as a tubular structure having a to diameter of 7-14 mm which is capable of being compressed and folded into a 6-9 French (F) sheath. Alternatively, the radially expandable device can be pre-shaped as a tubular structure having a diameter of 2-5 mm which can be crimped into a 6-9 F sheath and expanded (plastically) to a diameter of 7-14 mm The tubular structure can be 15-80 mm long, preferably, 20-70 mm long, more preferably 30-50 mm long.

The tubular structure can be covered with a graft on its external or internal (luminal) surface. The graft can cover the entire circumference and length of the tubular structure or a portion thereof (e.g. less than 360 degrees around the tubular structure and/or a portion of its length). The graft can be fabricated from Dacron, PTFE, polyurethane and like materials and glued, stitched or cast onto a unitary sub frame (e.g. stent or strut-interconnected rings) or a sub frame including two discrete rings.

Since closure requires sealing of the access site hole, the delivery catheter and the radially expandable device are configured such that the radially expandable device spans the vascular access site to thereby seal the access site hole following positioning thereof via the delivery catheter.

To achieve such functionality, both the delivery catheter and the radially expandable device are designed and configured for T-shaped deployment within the lumen of the blood vessel. Such T-shaped deployment is achieved by attaching the delivery catheter (and guide wire threaded therethrough) through an aperture provided in a mid section of the radially expandable device. This ensures that when the device is delivered through the access site hole and deployed via the delivery catheter, the portions of the device disposed on either side of the aperture, flank the access site hole, while the aperture aligns with the access site hole.

The above described configurations of the radially expandable device and delivery catheter which are collectively referred to herein as system 10 are described in more detail below with respect to the accompanying drawings.

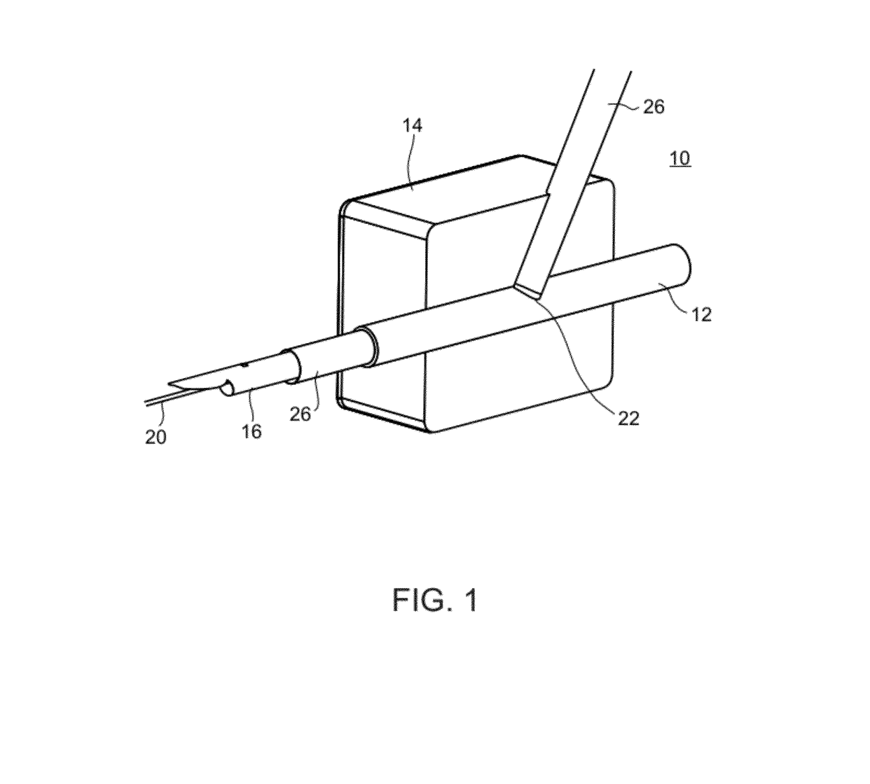

FIGS. 1-8 illustrates system 10 through stages of deployment within an artery 12 surrounded by tissue 14. System 10 includes a delivery catheter and a radially expandable device which in the example illustrated below is a stent-graft.

Referring now to FIG. 1 which illustrates delivery catheter 16 mounted over guide wire 20, and within a standard introducer sheath 26 typically used for percutaneous procedures; guide wire 20, introducer sheath 26 and delivery catheter 16 to are delivered through tissue 14 and access site hole 22 to the lumen of artery 12. Typically, an access site hole 22 is formed using a needle and guide wire 20 is then threaded therethrough. The needle is then removed and introducer sheath 26 is mounted over guide wire 20 and delivered through access site hole 22, widening it in the process to the desired diameter. Delivery catheter 16 can then be introduced into artery 12 over guide wire 20 and within introducer sheath 26.

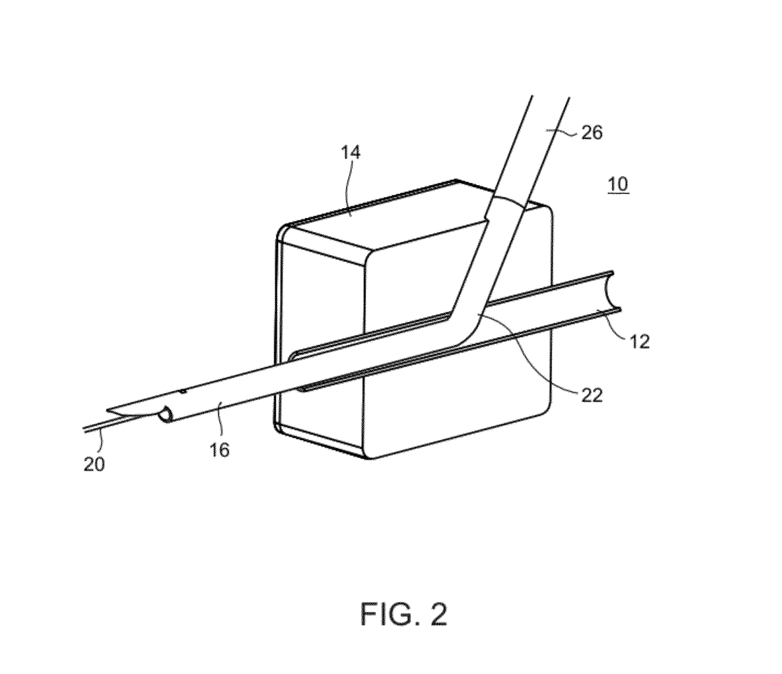

Following positioning of delivery catheter 16 within artery 12, introducer sheath 26 is pulled back through access site hole 22 and out of tissue 14 (FIG. 2).

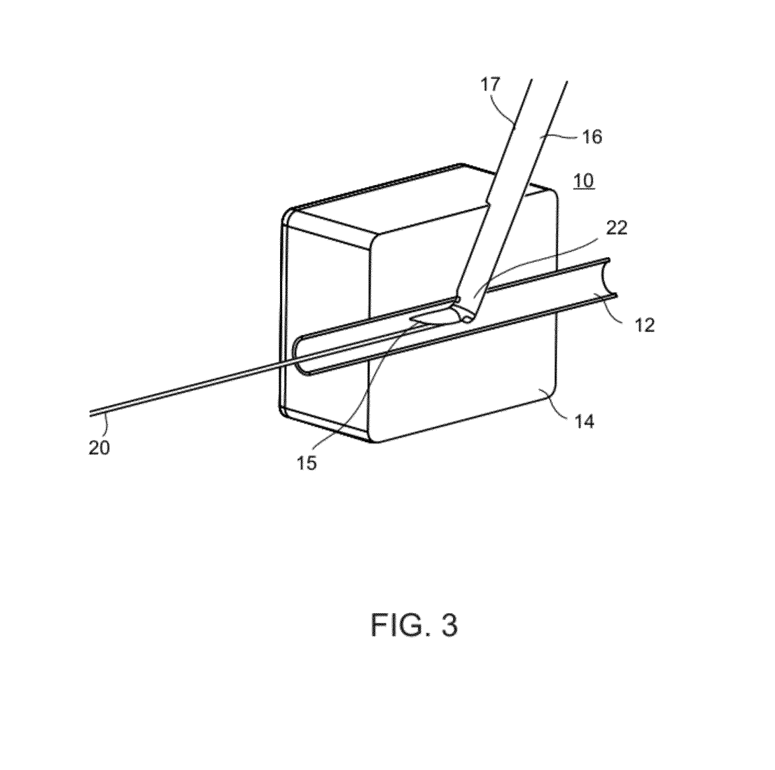

Delivery catheter 16 is then pulled back (in a proximal direction) until an end portion 15 thereof is positioned against the inner artery wall at access site hole 22 (FIG. 3). Distal portion 15 is pivotally attached and thus can angle and align with the longitudinal axis of artery 12 to facilitate deployment of expandable device 18 described below. Delivery catheter also includes an inspection hole 17 for indicating correct positioning of distal portion 15 of delivery catheter 16 within artery 12. Such an indication can be provided by stoppage of blood dripping from inspection hole 17 when delivery catheter is correctly positioned. To enable such functionality inspection hole 17 is fluidly connected to a conduit which terminates at a distal region of delivery catheter 16. When that distal region of delivery catheter is positioned within artery 12, the conduit communicates blood flowing through artery 12 to inspection hole 17, however, when delivery catheter is pulled back to a position in which that distal region is out of the blood flow, no blood is communicated to inspection hole 17 and as such dripping stops. Thus, by slowly pulling out delivery catheter 16 and watching for cessation of blood flow through inspection hole 17, one can correctly position delivery catheter 16.

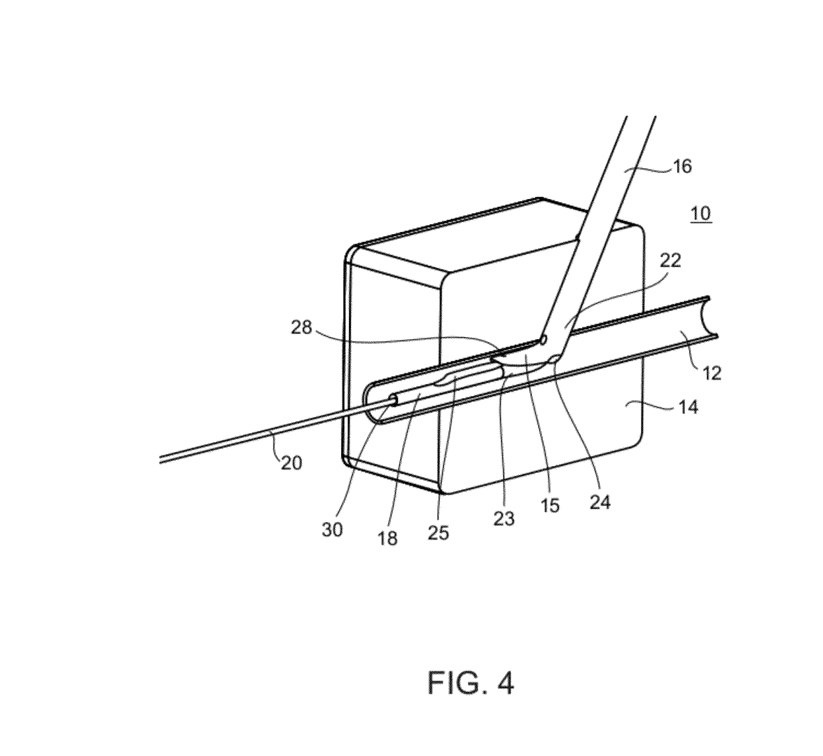

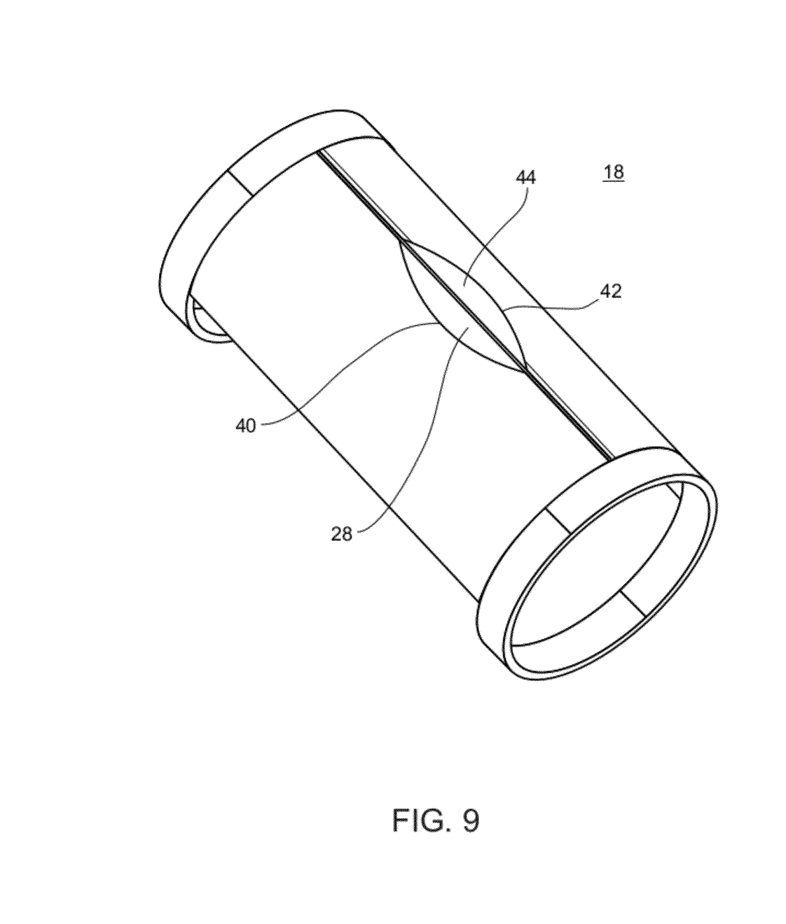

Delivery catheter 16 includes radially expandable device 18 (also referred to herein as device 18) within a lumen 24 thereof (device 18 shown in FIGS. 4-8 and separately shown in FIG. 9). In the present configuration of system 10, guide wire 20 is threaded within a positioning tube 25 which runs through lumen 24 (within internal tube 23, described below) of delivery catheter and through an aperture 28 of device 18; guide wire exits system 10 through a distal end 30 of device 18. When pushed out of lumen 24 of delivery catheter 16, device 18 remains connected to delivery catheter 16 (via the tube describe above) at a mid portion thereof and forms a T-shaped end with delivery catheter 16 as is further detailed below.

Delivery catheter 16 includes internal tube 23 for deploying radially expandable device 18. Internal tube 23 (pusher tube) is assembled over positioning tube 25 (both within lumen 24) such that when internal tube 23 is advanced distally, it pushes device 18 with attached positioning tube 25 out of the outer housing of delivery catheter 16 (FIG. 4). The distal portion of positioning tube 25 is preshaped with a 45-90 degree bend. When within tube 23, positioning tube 25 is held straight, however, when it is pushed out along with device 18 it assumes its preshaped (bent) position, thereby facilitating correct positioning of device 18.

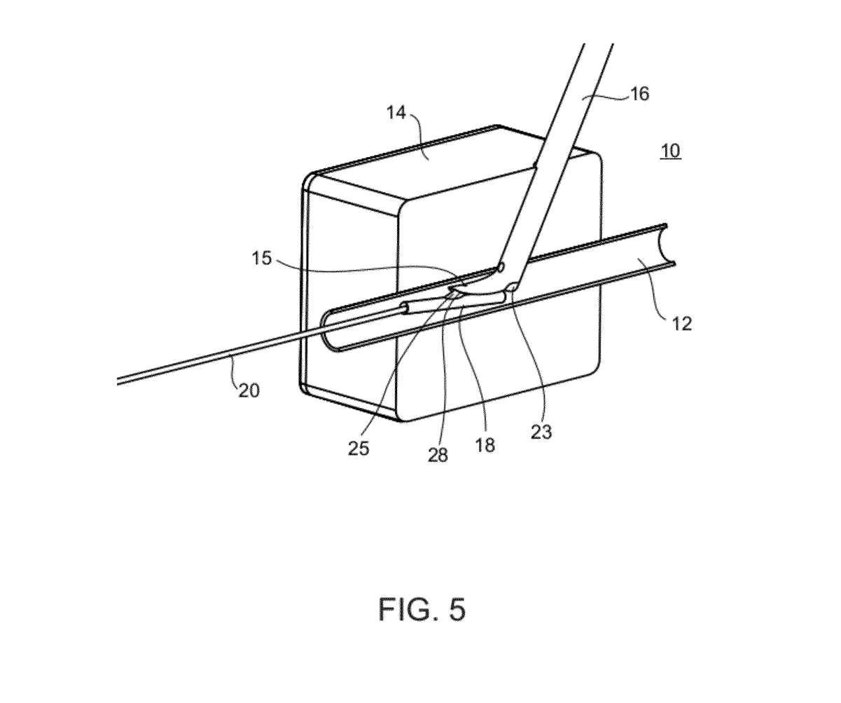

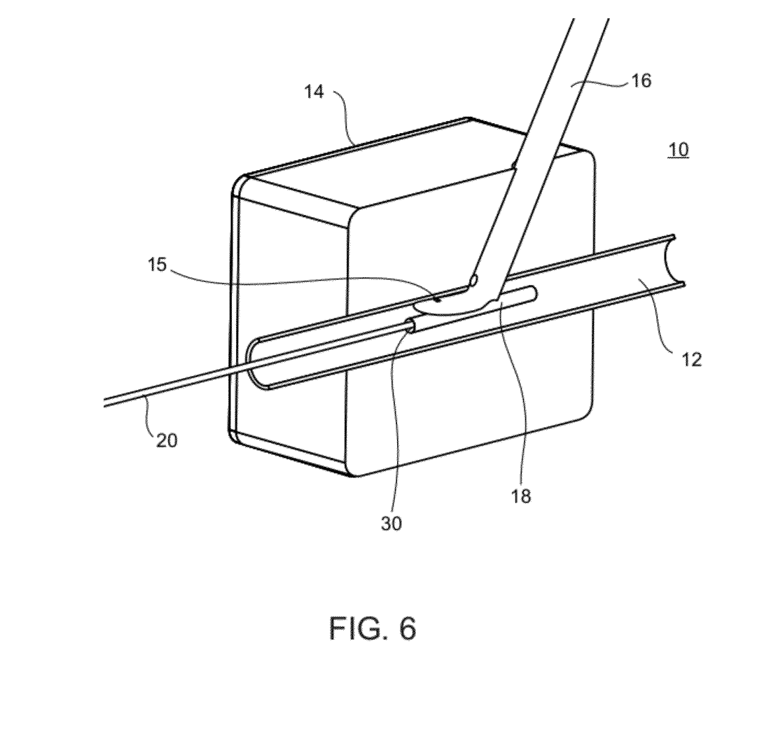

Once radially expandable device 18 is exposed, it is maneuvered into a T-position by pulling positioning tube 25 proximally (FIGS. 5-6). Positioning tube 25 also serves to route guide wire 20 through delivery catheter 16 and into radially expandable device 18 through aperture 28.

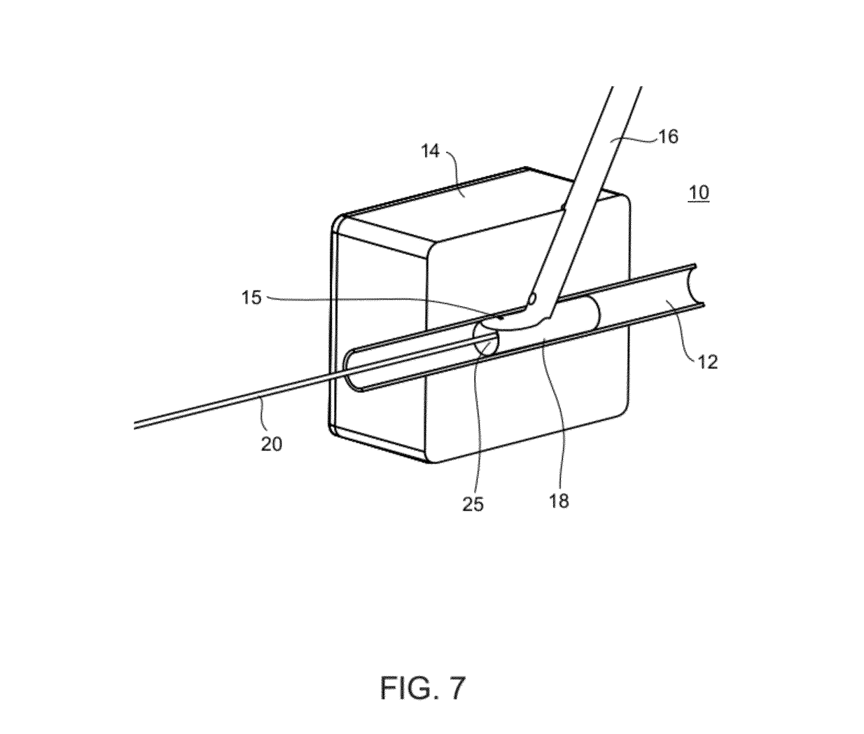

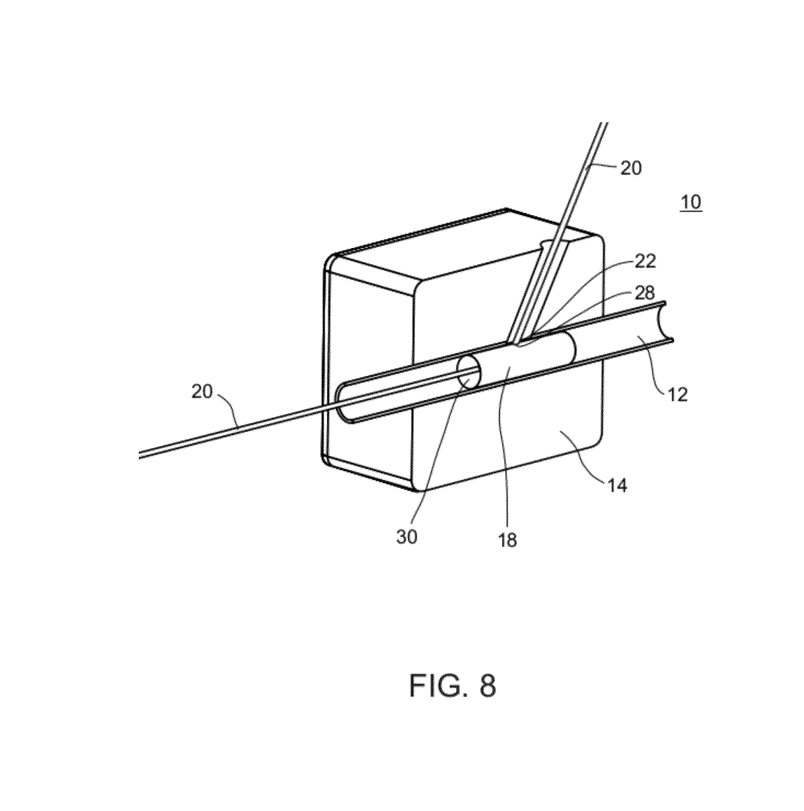

When in the T-position, aperture 28 of device 18 is aligned with access site hole 22 and enables expansion of device 18 in the correct position maintaining alignment between aperture 28 and access site hole 22. Device 18 is then expanded using one or more expansion mechanisms as described below (FIG. 7). Once delivery catheter 16 and introducer sheath 26 are removed, device 18 maintains its expanded position across access site hole 22 (FIG. 8) with guide wire 20 running through tissue 14, aperture 28 and out of distal end 30 of device 18 and into artery 12. Aperture 28 is preferably designed to close around guide wire 20 so as to minimize or prevent blood leakage. Self-sealing features of aperture 28 are further described hereinbelow.

Device 18 can be actively expanded or it can be self expanding. For Example, a stent graft configuration of device 18 can be wrapped by a thin sheath (0.025-0.2 mm thick) of nylon, PTFE or Dacron and maintained at a diameter of 1-3 mm in a non-expanded (compressed) state. The sheath is glued mid length to positioning tube 25 and is locked over device 18 via a wire (Nitinol, silk or other). The lock/stitching wire extends from the sheath/device 18 into the catheter and out to an actuating handle attached proximally to delivery catheter 16. Puling the wire releases the sheath and enables expansion of device 18. Several locking options are contemplated. The locking wire can be stitched into the sheath along its length or it can glued thereto. In any case, to deployment can be gradual along the length of device 18 (e.g. gradual expansion from one end to the other) or it can be stepwise, where one end (e.g. distal end of device 18) is deployed via pulling of locking wire to a first position following which further pulling of the locking wire releases the other end of device 18.

Since the locking wire connects to device 18 at a mid region (area of aperture 28) it can also be configured to separated pull 2 ends of two locking wires thereby opening the wrapping sheath from both ends simultaneously.

Expansion can also be effected using a balloon. In such an approach, a balloon is used to tear open the wrapping sheath described above. Once the balloon is inflated device 18 expands, applies a radial force on the wrapping sheath and rips it open at predefined point or points along a predefined line along the wrapping sheath (a precut notch or a series of small holes). When the wrapping sheath is fully ripped open (along its longitudinal axis) device 18 expands to its final dimensions.

Following deployment, the balloon is deflated and is pulled out along with along with the wrapping sheath (both are connected to positioning tube 25) through aperture 28 and delivery catheter 16, aperture 28 can then self-seal as described below.

A balloon expanded configuration of device 18 is also envisaged. In such a configuration, device 18 is fabricated in a compressed state and is actively expanded (via plastic deformation) using a balloon.

A stainless steel or Cobalt Chromium stent graft is positioned over a balloon mounted and attached to a fluid filling tube 25 within delivery catheter 16. Device 18 in a compressed state (1-3 mm in diameter) is crimped over the balloon with the fluid filling tube routed through aperture 28. Inflating the balloon to 7-14 mm in diameter will plastically deform device 18 to the desired expanded size. Once device 18 is deployed, the balloon is deflated and delivery catheter 25 with enclosed balloon are pulled out through aperture 28 and access site hole 22.

Housing of delivery catheter 16 is constructed as a tube having a lumen which includes device 18 and tubes 23 and 25 in a coaxial arrangement. The housing and tubes can be molded from any suitable material, examples include polymers, alloys, ceramics and the like.

Once delivery catheter 16 and guide wire 20 are removed from the body, aperture 28 can either self seal or be sealed using an adhesive, a patch or a combination thereof.

Several self-sealing mechanisms can be used to partially or fully close aperture 28.

One sealing configuration can employ a wire frame oval as aperture 28 (oval arcs indicated by 40 and 42 in FIG. 9) which is heat treated to a “normally closed” position in which opposing arcs 40 and 42 of the oval cross each other thereby minimizing area 44. When device 18 is assembled within delivery catheter 16 such that positioning tube 25 is fed through aperture 28, arcs 40 and 42 are forced apart thereby opening aperture 28. Once delivery catheter is pulled out of the body, arcs 40 and 42 of aperture 28 close over guide wire 20, pulling out guide wire 20 allows final closure of aperture 28.

Aperture 28 designed for partial sealing can close to a predetermined point and then be completely sealed using an adhesive, pad, patch or a combination thereof, or it can be sealed via coagulation induced by a coagulant or manual pressure. In any case, closure is preferably effected using a mechanism that would allow for artery re-entry through aperture 28.

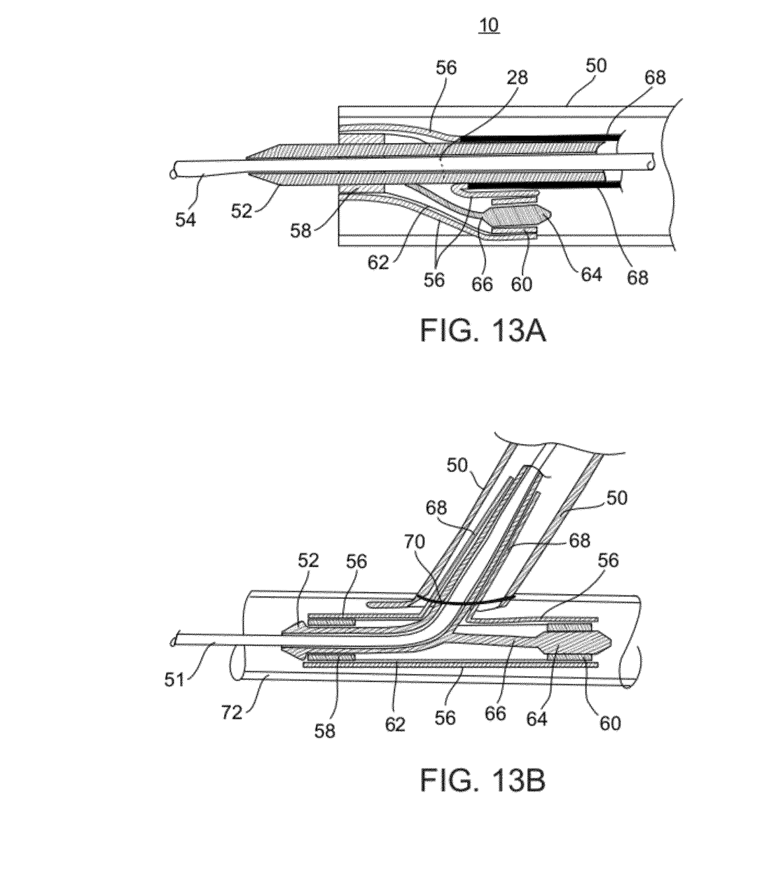

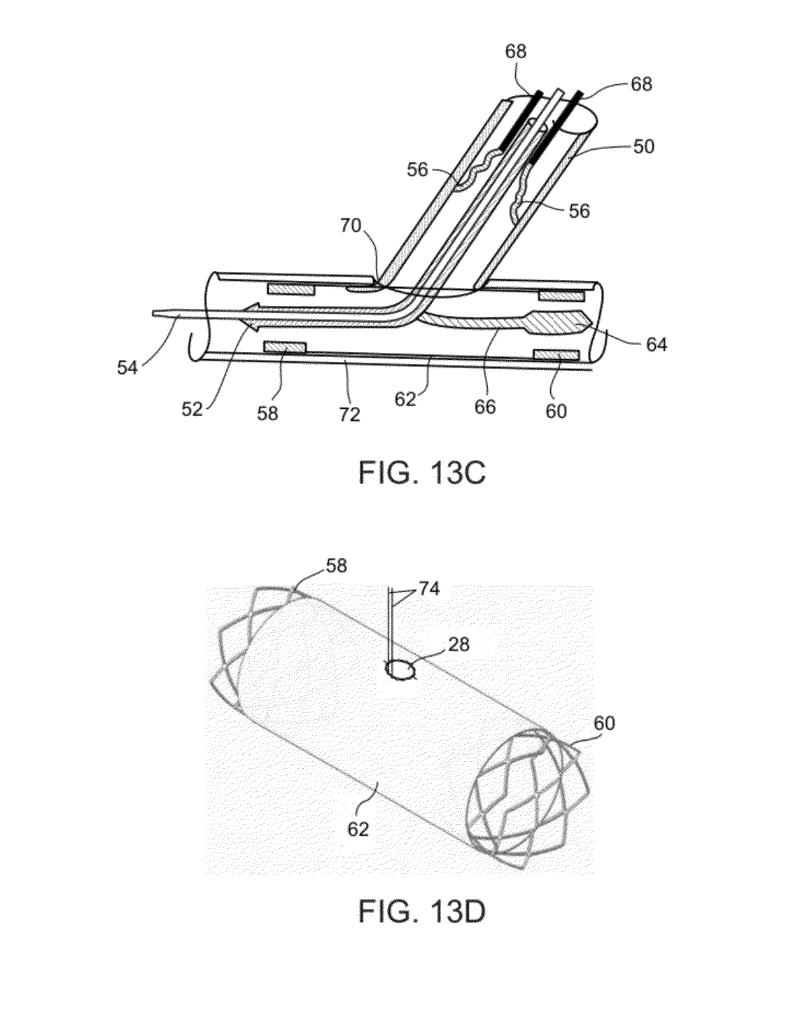

FIGS. 13A-C illustrate another embodiment of system 10 as operated through the various stages of deployment.

System 10 packed with device 18 and ready for use is shown in FIG. 13A. This embodiment of system 10 includes an external sheath 50 which is delivered as is or through a standard delivery sheath (not shown) into a blood vessel (shown in FIGS. 13B-C)) and an internal sheath 52 which is mountable over a guidewire 54. System 10 further includes a device locking sheath 56 which locks device 18 in a compressed state around internal sheath 52 and within external sheath 50. Device 18 (separately shown in FIG. 13D) includes a proximal stent-like ring 58 and a distal stent-like ring 60 interconnected by a graft 62. Proximal ring 58 is compressed over internal sheath 52, while distal ring 60 is compressed over a distal portion 64 of ‘boom’ arm 66. Boom arm 66 and mounted distal ring 60 are held against internal sheath 52 and held in position by external sheath 50.

As is further described below with reference to FIG. 13D, graft 62 includes an aperture 28 (noted by dotted line), through which internal sheath 52 is routed. Thus, device 18 is packed within external sheath 50 with proximal ring 58 disposed (compressed) around internal sheath 52 and distal ring 60 (compressed) disposed adjacent to internal sheath 52.

Device locking sheath 56 is connected (e.g. glued, sutured) to a lock removal mechanism 68 which functions in removing device locking sheath 56. Lock removal mechanism can be realized by a pair of pull wires, a sheath and the like.

System 10 as shown in FIG. 13A is inserted through an access site 70 and into an artery 72 over a guidewire 54. External sheath 50 is then pulled back (out) until blood outflow is detected. One approach for detecting blood outflow (and thus providing an indication of external sheath 50 position) is via use of side holes in an intermediate sheath or tube disposed between external sheath 50 and internal sheath 52. Such side holes would be covered by external sheath 50 and thus no blood will flow out through such holes. However, pulling back external sheath 50 and exposing such side holes will lead to blood outflow and an indication of system 10 position within the artery. Alternatively, an indication of the correct positioning of system 10 can be as described with respect to FIG. 3 above.

External sheath 50 is then held in position and the components housed within external sheath 50 are advanced further into artery 72. As result, boom arm 66 which was held against internal sheath 52 by external sheath 50 is released, such that distal ring 60 now assumes a co-linear position with proximal ring 58 at this stage, system components are pulled back to allow distal ring 60 to be located distally to the entry site while proximal ring 58 is located proximally to the entry site (FIG. 13B). Lock removal mechanism 68 is then pulled back and out releasing device locking sheath 56 (tearing it) from device 18 and thereby expanding proximal ring 58 and distal ring 60 (stepwise or concomitantly). Device locking sheath 56 can have a tear pattern (formed by perforations) along which it tears when pulled.

Internal sheath 52 and external sheath 50 are then completely removed from artery 72 and aperture 28 is using a self closing wire frame oval (not shown) which is glued or fastened to the graft material at the site of aperture 28. Alternatively and preferably, aperture 28 is partially or fully closed or via prepositioned sutures (further described hereinbelow with respect to FIG. 13D). Guidewire 54 is then removed from the artery and aperture 28 completely sealed via these sutures or by an adhesive or the like.

It will be appreciated that although release of device locking sheath 56 and expansion of proximal ring 58 and distal ring 60 is effected via release mechanism 58 which pulls, tears and removes device locking sheath 56, other release mechanisms such as balloons mounted over internal sheath 52 (under proximal ring 58) and boom arm 66 (under distal ring 60) can also be used to tear and release device locking sheath 56.

As is mentioned above, this embodiment of system 10 includes a device 18 which is formed from a sleeve interconnecting two opposing stent-like rings.

As shown in FIG. 13D, device 18 includes proximal ring 58, distal ring 60 and graft 62. Graft 62 includes aperture 28 which is positioned along a length of graft 62 preferably at a midway point between proximal ring 58 and distal ring 60.

Proximal ring 58 and distal ring 60 can be made from stainless steel, Nitinol and the like by laser cutting a stent pattern from a tube having a length of 6-12 mm (along longitudinal axis of device 18). Rings 58 and 60 can be 2-3 mm in diameter when compressed and 7-12 mm in diameter when expanded. The total length of device 18 (distance between outer edges of rings 58 and 60) can be 20-40 mm Graft 62 can be a rolled sheet or a mandrel formed graft made from Dacron, ePTFE and the like. Graft 62 can be glued, stapled or sutured onto rings 58 and 60. Aperture 28 can be 2-4 mm in diameter with a capability of elastically expanding to accommodate devices/sheaths having diameters of 8 mm or more. Aperture 28 can be reduced to 1 mm or less (0 mm) in diameter via suturing as described below.

FIG. 13D also illustrates an alternative aperture 28 closing approach. In this configuration of device 18, closure is preferably effected using one or more sutures 74 that are prepositioned around aperture 28. The suture or sutures can be threaded through the graft material in a purse string configuration or any other configuration which enables access through aperture 28 and simple closure following the procedure [e.g., by pulling one or more ends of the suture(s) outwardly].

The device 18 configuration shown in FIG. 13D provides several advantages, especially when used in femoral access site closure:

(i) stents positioned in a femoral arteries can be exposed to bending forces (e.g. caused by leg movement) that can potentially lead to breakage and stent failure. Since only a small portion of device 18 (the rings) is stent-like, it is less susceptible to such forces than a full stent body.
(ii) two independent anchoring regions reduce movement (creeping) of the device to under the forces of pulsatile blood flow.
(iii) since in femoral closure the present device is positioned near the pelvic joint leg movement may lead to cyclic stress. A short device will be less exposed to such stress then a longer device. In fact, a device having the length of the present device will be exposed to little or no stress. In addition, since the present device includes two narrow rings interconnected by graft material, it will not be susceptible to the “bending” fatigue characteristic of full stent implants.

As is further described in Example 2 of the Examples section which follows, a preferred configuration of such a device 18 includes self expanding super-elastic alloy rings each capable of applying a radial force of at least 0.8-2N when expanded against the inner arterial wall (intima). This ensures that device 18 does not migrate under the pulsatile flow of blood in the artery while it also ensures that non-symmetrical compression forces applied to each or any of the expanded rings do not lead to non-reversible buckling (inward collapse of a sector) without elastic rebound.

Device 18 can include a radio opaque marker or markers surrounding aperture 28, such markers would allow identification of aperture 28 once embedded in the artery using imaging techniques. Such identification could be used for re-entry if necessary.

It will be appreciated that although the present system is described herein with respect to vascular access site closure. It can also be used for closure of other tissue opening of other tubular vessels or structures, such as for example, a urethra, ureters, portions of the GI tract, or for delivery of a stent-graft device into a tubular vessel, such as a blood vessel, for purposes not related to access site closure.

As used herein the term “about” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### T-Graft Deployment-Feasibility

A feasibility test was designed in order to illustrate the usability of the deployment approach described herein (T-deployment). A tubular-shaped element simulating a wrapped stent graft was connected to delivery catheter at a mid-portion of the element. A silicon tube simulating an artery with an access site hole was wrapped in a foam block simulating surrounding tissue and was used as a tissue phantom.

System

The delivery system included a 15 F external sheath with a 12 F pusher tube. The radially expandable device (wrapped stent) was a tubular element 3 mm in diameter and 30 mm in length. A 6 F pigtail diagnostic catheter was inserted through a side hole in the tubular element and glued thereto to create the functionality for the required T-shaped delivery. The system was assembled by threading the 12 F pusher over the 6 F catheter. Both were inserted into the 15 F catheter (functioning as the catheter housing) while positioning the tubular element in line with the 12 F pusher.

Procedure

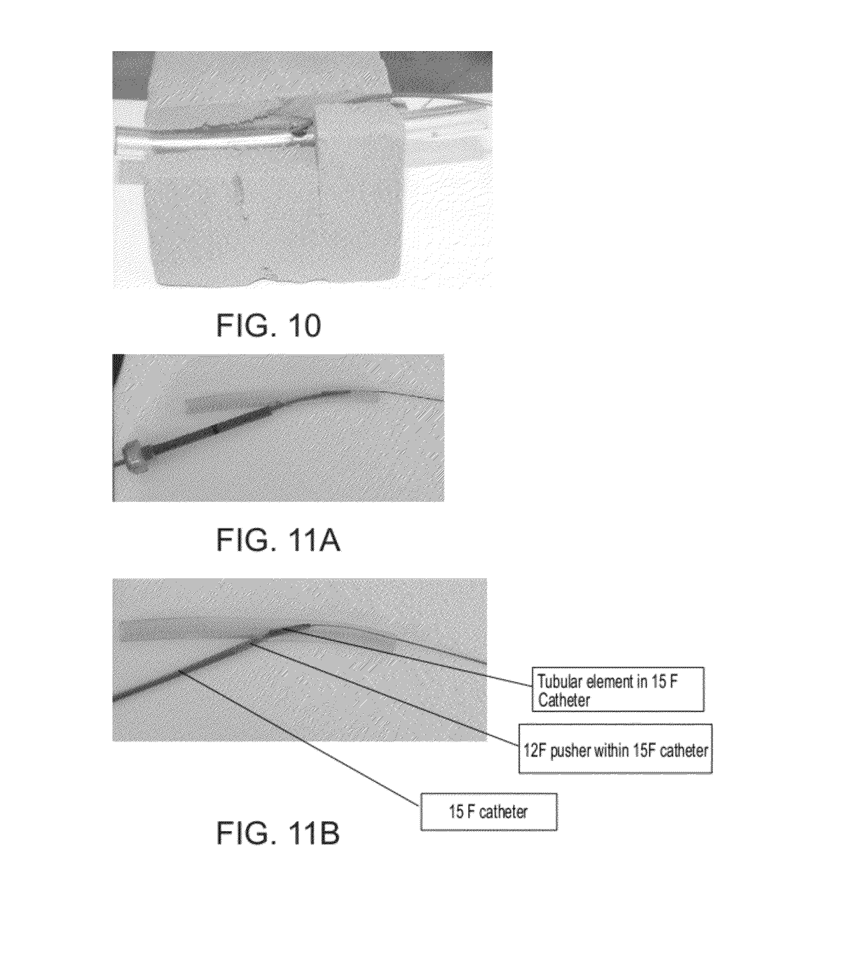

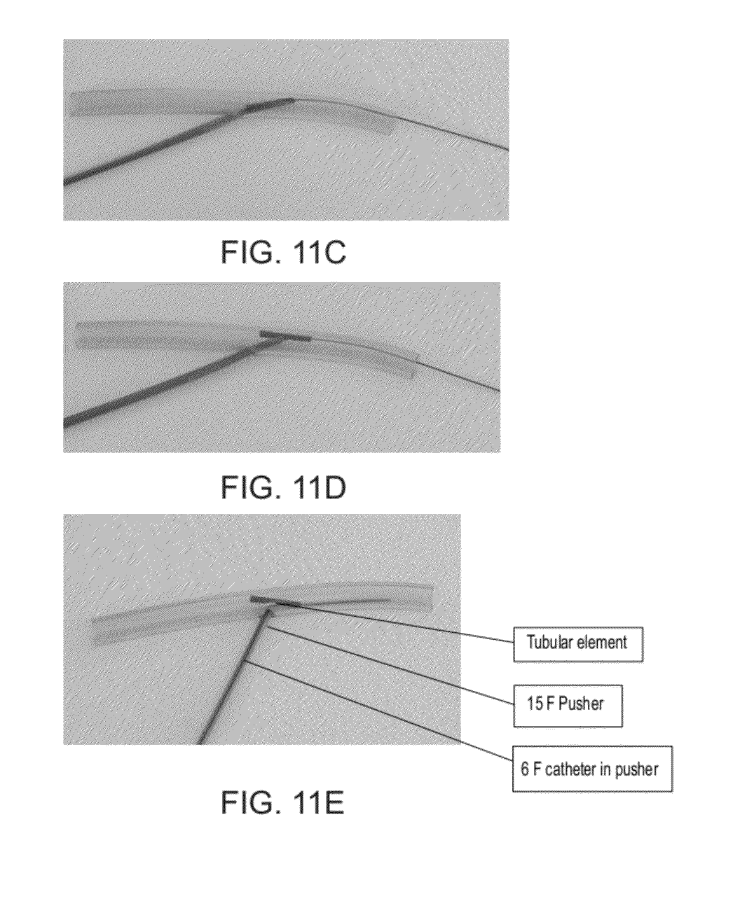

A 10 mm diameter silicone tube simulating a femoral artery was positioned within a hole drilled through a foam block simulating surrounding tissue (FIG. 10). A 30-45 degree 8 mm diameter entry hole (access site hole) was drilled through the foam block and into the silicone tube. A guide wire was threaded into the silicone tube and the assembled system including the catheter outer housing (15 F catheter) containing the 12 F pusher tube, the 6 F catheter and the tubular element (connected to the 6 F pigtail catheter) was positioned within a 26 F introducer sheath and mounted over the guide wire (FIG. 11A, mounted system shown without foam block).

Deployment of the tubular element procedure was carried out while the silicone tube was positioned within the foam block. However, for illustrative purposes the foam block was removed and the procedure repeated in order to clearly show the stages of deployment of the tubular element (FIGS. 11B-E).

Following positioning of the system within the silicone tube, the introducer sheath was removed and the 15 F catheter was pulled back to a position near the access site hole (FIG. 11B). While the 15 F was held in position, the 12 F pusher was advanced distally until the tubular element was pushed completely out of the 15 F catheter (FIG. 11C). The pusher and 6 F catheter along with attached tubular element were then pulled back (proximally) to thereby trap the tubular element in a t-position (FIG. 11D). The 15 F catheter along with the pusher and attached pigtail catheter and tubular element (in the t-position) were then pulled back (proximally) to align the tubular element with the access site hole and the 15 F was removed (FIG. 11E).

### Example 2

### Stent-Graft

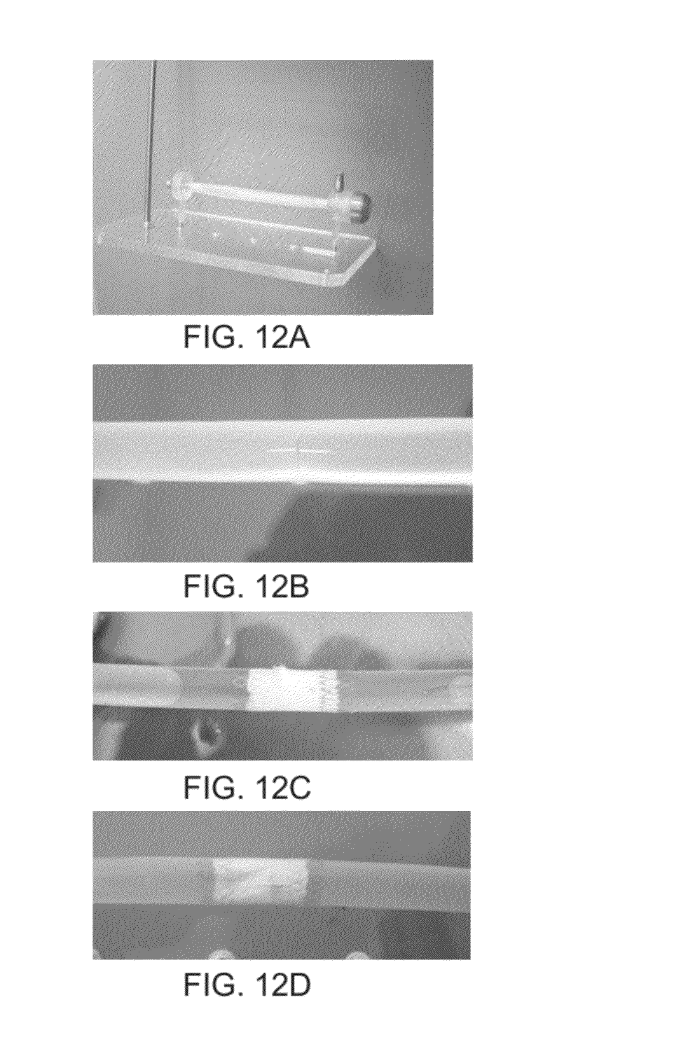

A stent-graft fabricated from two opposing stet-like rings interconnected via a tubular sheet cover (FIG. 13D) was fabricated and tested for sealing and structural integrity using a platform modeling flow in an artery (FIG. 12A). The platform included a silicon tube simulating an artery (O.D. 10, I.D. 9 mm) and a non-pulsatile fluid pressure source for simulating blood pressure within the simulated artery (a number 5 Syringe, digital pressure meter).

Two stent-graft configurations were fabricated by stitching an ePTFE tube (Zeus, 0.415″ id×4 mil thick) over two discrete pre-shaped nitinol stents (rings) fabricated by laser cutting 3 mm od nitinol tube in two rows of 14 cells pattern. The first configuration stent-graft was heat treated to 10.5 mm diameter, 8 mm in length and 0.08 mm thick, the second configuration post treatment dimentions where 11.5 mm diameter, 7 mm in length and 0.11 mm thick.

The silicon tube was cut to simulate an access site (FIG. 12B), and the stent-graft was positioned within the silicone tube using a delivery device (not shown). The platform was then used to test:

(i) stent-graft delivery, positioning and expansion within the tube;
(ii) sealing of access site; and
(ii) stent-graft response to pressure.

### Procedure

Using a simple axial delivery system the device was located within a silicone tube and released under a pre-cut side hole in the silicone tube. The delivery system was removed and a syringe was used to inject water through the silicon tube, pressure was raised to 300 mm Hg and leakage through the side hole was monitored.

### Results

### Configuration 1

Sealing was obtained under fluid pressures of 300 mmHg. In this configuration, the Stent length to ID ratio is approximately 1:1 when expanded, while in the collapsed state, this ratio is 1:3. As a result when expanded within the artery, the stent distal side will achieve full I.D. only following total expansion and anchoring of the proximal side. This can lead to release instability and may also affect graft behavior.

### Configuration 2

Sealing was obtained under fluid pressures of 300 mmHg This configuration was designed in order to traverse the limitations of configuration 1. Thus, the radial force and radial kink stability was enhanced in order to improve device apposition and device release stability. The radial force of this configuration was increased by a factor of 1.37, while kink resistance was improved by a factor of 2.5. This led to an improved kink resistance and improved stability in delivery and deployment.

The device was further tested for stability against external compression forces designed to mimic the forced encountered in an artery, namely forces due to pulsatile flow of blood and movement of the patient (e.g. bending and muscle forces caused by limb movement). External forces applied to one of the rings lead to an inward and irreversible collapse of the ring (FIG. 12D).

Analyzing these results led to the conclusions, that in order to improve radial stability (against collapse) of the device, wall thickness of individual stent struts should be increased a factor of 2. This will result in a 2× increase in radial force and an 8× increase in kink resistance.

It is appreciated that certain features of the invention, which are, for clarity, to described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A system for closure of a vascular access site comprising:
(a) a radially expandable device sized and configured for positioning within a blood vessel; and
(b) a delivery catheter for:
(i) delivering said radially expandable device through the vascular access site; and
(ii) expanding said radially expandable device in a region within said blood vessel spanning the vascular access site, thereby at least partially closing said vascular access site.

2. The system of claim 1, wherein said radially expandable device is a stent graft.

3. The system of claim 1, wherein said radially expandable device is self expanding.

4. The system of claim 1, wherein said radially expandable device includes two opposing expandable rings interconnected via a sleeve.

5. The system of claim 4, wherein each of said two opposing expandable rings is less than 10 mm in width.

6. The system of claim 4, wherein said sleeve is fabricated from a tubular sheet of ePTFE.

7. The system of claim 1, wherein said delivery catheter includes a mechanism for expanding said radially expandable device.

8. The system of claim 7, wherein said radially expandable device includes a balloon and said mechanism is an inflation mechanism.

9. The system of claim 7, wherein said radially expandable device is compressed within a sheath and said mechanism is a sheath removal mechanism.

10. The system of claim 1, wherein said radially expandable device includes an aperture in a side wall along a length thereof, said aperture being for providing said delivery catheter access to a lumen of said expandable tubular body.

11. The system of claim 10, wherein said delivery catheter and said radially expandable device form a t-shape when said radially expandable device is positioned in said blood vessel.

12. The system of claim 10, wherein said delivery catheter engages said radially expandable device through said aperture, such that a guide wire can be threaded through said delivery catheter though a lumen of said radially expandable device and into a lumen of said blood vessel.

13. The system of claim 12, wherein said aperture is capable of at least partially closing when said delivery catheter is disengaged therefrom.

14-17. (canceled)

18. A method of at least partially closing a vascular access site comprising:
(a) positioning a radially expandable device having an aperture in a side wall along a length thereof within a blood vessel through the vascular access site; and
(b) aligning said aperture with said access site and expanding said radially expandable device within said blood vessel at a region spanning the vascular access site; and
(c) at least partially closing said aperture thereby at least partially closing the vascular access site.

19. The method of claim 18, wherein steps (a) and (b) are effected using a delivery catheter.

20. The method of claim 19, wherein said delivery catheter engages said radially expandable device through said aperture, such that a guide wire can be threaded through said delivery catheter though a lumen of said radially expandable device and into a lumen of said blood vessel.

21. The method of claim 19, wherein said delivery catheter includes a mechanism for expanding said radially expandable device.

22-27. (canceled)

28. The system of claim 4, wherein said delivery catheter includes an internal sheath and an external sheath, wherein said radially expandable device is packable within said external sheath with said first ring of said two opposing expandable rings being disposed around said internal sheath and said second ring of said two opposing expandable rings being disposed adjacent to said internal sheath.
